(19) United States
(12) Patent Application Publication
(10) Pub. No.: US 2024/0392382 A1
(43) Pub. Date: Nov. 28, 2024

(54) MUTATIONAL MARKERS IN PANCREATIC NEUROENDOCRINE TUMORS FOR USE IN STRATIFYING RESPONSE TO CAPECITABINE/TEMOZOLOMIDE

(71) Applicant: CEDARS-SINAI MEDICAL CENTER, Los Angeles, CA (US)

(72) Inventors: Andrew Hendifar, Santa Monica, CA (US); Patrick Lee, Los Angeles, CA (US)

(73) Assignee: CEDARS-SINAI MEDICAL CENTER, Los Angeles, CA (US)

(21) Appl. No.: 18/672,478

(22) Filed: May 23, 2024

Related U.S. Application Data
(60) Provisional application No. 63/468,721, filed on May 24, 2023.

(51) Int. Cl. C12Q 1/6886; A61K 31/495; A61K 31/7064

(52) U.S. Cl. CPC C12Q 1/6886; A61K 31/495; A61K 31/7064; C12Q 2600/106; C12Q 2600/118; C12Q 2600/156

(57) ABSTRACT

We describe a new genomic signature (MEN1 mut/DAXX wt) that correlates with PNET response to CAPTEM therapy. MEN1-mut/DAXX-wt status correlates with longer progression-free survival on CAPTEM in patients with pancreatic neuroendocrine tumors, relative to patients with MEN1-wt or DAXX-mut.

### CROSS-REFERENCE TO RELATED APPLICATIONS

This application includes a claim of priority under 35 U.S.C. § 119(e) to U.S. provisional patent application No. 63/468,721, filed May 24, 2023, the entirety of which is hereby incorporated by reference.

### FIELD OF INVENTION

This invention relates to genomic mutation profile for selecting chemotherapy and/or predicting responsiveness to selected chemotherapy in patients with pancreatic neuroendocrine tumors.

### BACKGROUND

In the past, patient received treatments for their neuroendocrine tumors based on physician discretion. With the approval of SOMATULINE® (lanreotide) for somatostatin receptor expressing neuroendocrine tumors, the PET/CT gallium-68 dotatate scan helps stratify patients to whether they can receive somatostatin analog therapy and radionuclide therapy with LUTATHERA® (Lutetium Lu 177 dotatate). Otherwise, there is no predictive blood test or tumor assay that helps clinicians or patients select or personalize therapeutic approaches. This is despite the fact that pancreatic neuroendocrine tumors are quite heterogeneous based on histology, functionality, grade, and differentiation.

It is an object of the present invention to provide methods of treatment for neuroendocrine tumors, e.g., pancreatic neuroendocrine tumors, based on mutation status of selected genes.

It is another object of the present invention to provide methods of stratifying patient's response or treatment outcomes to therapy for pancreatic neuroendocrine tumors based on mutation status of selected genes.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide methods of treating a subject with pancreatic neuroendocrine tumor (PNET), which include: administering an effective amount of a composition comprising a combination of capecitabine or a derivative of capecitabine and temozolomide or a derivative of temozolomide to the subject detected with mutated MEN1 and wild-type DAXX in a tumor sample of the subject. In some embodiments, the tumor sample comprises a pancreatic tumor sample.

Various embodiments provide methods for selecting a subject having PNET for CAPTEM therapy and treating the subject, and the methods include: detecting mutated MEN1 and wild-type DAXX in a tumor sample of the subject, and administering the CAPTEM therapy to the subject, wherein the CAPTEM therapy comprises a combination of an effective amount of capecitabine or a derivative/metabolite of capecitabine (e.g., fluorouracil) and an effective amount tof temozolomide or a derivative of temozolomide (e.g., dacarbazine).

Various embodiments provide methods for treating a subject having PNET, who has mutated MEN1 gene in a tumor sample of the subject, and the methods include detecting presence or absence of mutation in DAXXgene in a tumor sample of the subject, and administering a CAPTEM therapy to the subject if the DAXXgene is wildtype, or administering another treatment other than or in addition to the CAPTEM therapy to the subject if the DAXX gene has a mutation compared to wildtype DAXX. In some embodiments, another treatment includes but is not limited to ¹⁷⁷Lu-Dotatate, surgery, hormone therapy, hepatic arterial occlusion or chemoembolization.

In various aspects, a subject with the mutated MEN1 and the wild-type DAXX in his/her PNET tumor is likely to have a longer progression-free survival period in response to CAPTEM therapy than a control subject with the PNET but with wild-type MEN1 or mutated DAXX.

In some embodiments, a subject detected with mutated MEN1 and wildtype DAXX in his/her PNET sample, or who is responsive to CAPTEM therapy, also has mutated ATRX gene and/or mutated PTEN gene in the tumor sample.

In some embodiments, presence or absence of mutations in selected genes are detected by performing next-generation sequencing. In some embodiments, DNA, RNA, especially mRNA, or both of respective genes are sequenced to identify presence or absence of mutation.

In various aspects, the subject is a human.

Various embodiments provide methods of assaying a biological sample from a subject with pancreatic neuroendocrine tumor (PNET), and the methods include performing gene sequencing for mRNA (or DNA, or RNA) of one or more genes comprising MEN1, DAXX, ATRX, and PTEN in the biological sample, and detecting presence or absence of a mutation in each of the one or more genes, wherein the biological sample comprises a tumor cell or tissue of the PNET.

In various aspects, the one or more genes comprises the MEN1 and DAXX. In further embodiments, the methods include selecting the subject for a CAPTEM therapy, wherein the subject is detected with a presence of mutated MEN1 and an absence of mutated DAXX, and wherein the CAPTEM therapy comprises a combination of capecitabine or a derivative of capecitabine and temozolomide or a derivative of temozolomide.

In various aspects, the subject has a mutation in the MEN1 gene, and the methods include performing of gene sequencing of one or more of DAXX, ATRX, and PTEN of the subject.

In further embodiments, the methods include providing prognosis to a subject receiving a CAPTEM therapy, and/or determining if the subject is likely to respond to the CAPTEM therapy, wherein the subject is prognosed with a longer progression-free survival period after receiving the CAPTEM therapy and/or the subject is indicated to be better responsive to the CAPTEM therapy if the subject is detected with the presence of mutated MEN1 and wild-type DAXX, compared to a first control subject with the PNET but having wild-type MEN1 and/or mutated DAXX in a PNET sample of the first control subject; or wherein the subject is prognosed with a shorter progression-free survival period after receiving the CAPTEM therapy and/or the subject is indicated to be less responsive to the CAPTEM therapy if the subject is detected with wild-type MEN1 and/or the presence of mutated DAXX, compared to a second control subject with the PNET but having mutated MEN1 and wild-type DAXX in a PNET cell or tissue of the second control subject.

Further embodiments provide methods of treating a subject having pancreatic neuroendocrine tumor, wherein the subject is detected with wild-type MEN1 gene and/or mutated DAXX gene in a tumor sample, the method comprising: performing surgery to remove the tumor, administering ¹⁷⁷Lu-Dotatate, providing hormone therapy, and/or performing hepatic arterial occlusion or chemoembolization, to the subject detected with the wild-type MEN1 gene and/or the mutated DAXX gene. In further aspects, for a subject having PNET and detected with the wild-type MEN1 gene and/or the mutated DAXX gene, the method discontinues or does not include administering a CAPTEM therapy to the subject.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A depicts mutational differences between PNET Subgroups which are distinguished by MEN1-mutated/DAXX-wildtype status. ATRX mutations were more common in MEN1^{mut}/DAXX^{wt} tumors versus other next-generation sequencing (NGS) profiles from patients with PNET. FIG. 1B is a bar chart showing mutation frequencies of various genes across all PNET cases (N=95) in Example 1.

FIGS. 2A and 2B are Kaplan-Meier curves depicting exploratory PFS comparisons on 1^{st} (2A) or 2^{nd} (2B) line CAPTEM between PNET subgroups defined by NGS as having MEN1-mutated/DAXX-wildtype (MEN1mut/DAXXwt) status or not having the MEN1mut/DAXXwt (i.e., MEN1 wild type or DAXXmut). Median progression-free survival estimates with 95% confidence intervals for each PNET subset are described above the Kaplan-Meier curves for 1st line (2A) or 2nd line (2B) CAPTEM.

FIG. 3 depicts correlation of genetic alterations with time (days) on CAPTEM treatment in patients with PNETs. 860 patients with NETs were reviewed, and among which 114 patients were treated with CAPTEM. Out of the 114 patients, 41 had tumor NGS results, including 19 pNETs, 8 lung, 4 small/large bowel, 4 paraganglioma, and 6 others.

FIG. 4 depicts the effect of mTOR pathway alterations on the time (days) on CAPTEM treatment in patients with PNETs. Lower panel y-axis represents percentage of patients on therapy. Mutations to PTEN (n=2), TSC1 (n=3), TSC2 (n=1), MTOR (n=1), or NF1 (n=1). 860 patients with NETs were reviewed, and among which 114 patients were treated with CAPTEM. Out of the 114 patients, 41 had tumor NGS results, including 19 pNETs, 8 lung, 4 small/large bowel, 4 paraganglioma, and 6 others.

FIG. 5 depicts the relation of MTOR Status to other clinicopathologic factors. 860 patients with NETs were reviewed, and among which 114 patients were treated with CAPTEM. Out of the 114 patients, 41 had tumor NGS results, including 19 pNETs, 8 lung, 4 small/large bowel, 4 paraganglioma, and 6 others.

FIG. 6 depicts association of clinicopathologic factors with time on CAPTEM treatment. 860 patients with NETs were reviewed, and among which 114 patients were treated with CAPTEM. Out of the 114 patients, 41 had tumor NGS results, including 19 pNETs, 8 lung, 4 small/large bowel, 4 paraganglioma, and 6 others. Time refers to the period from initial CAPTEM treatment till death, disease progression, or change of therapy.

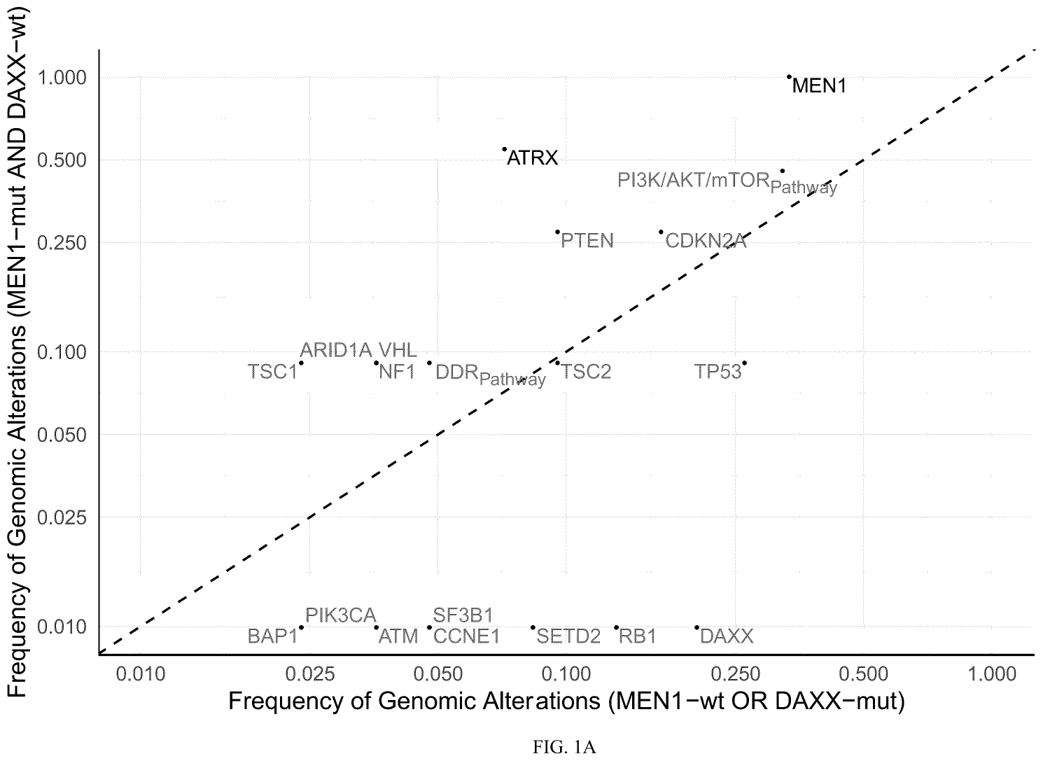

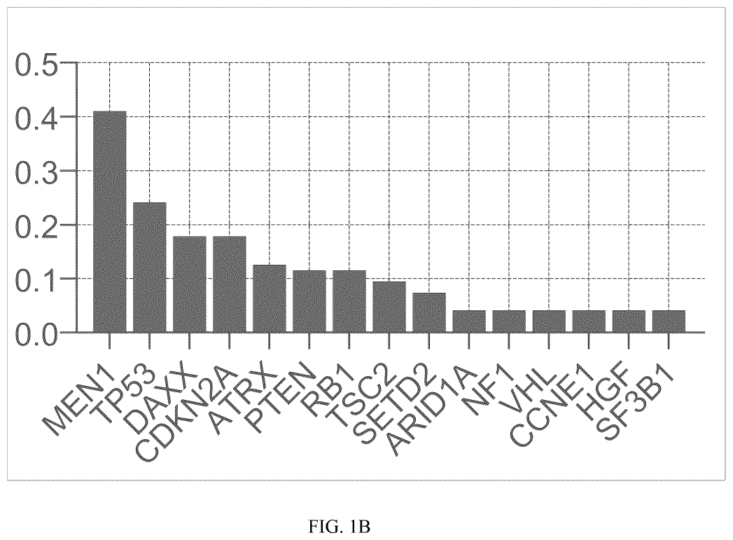

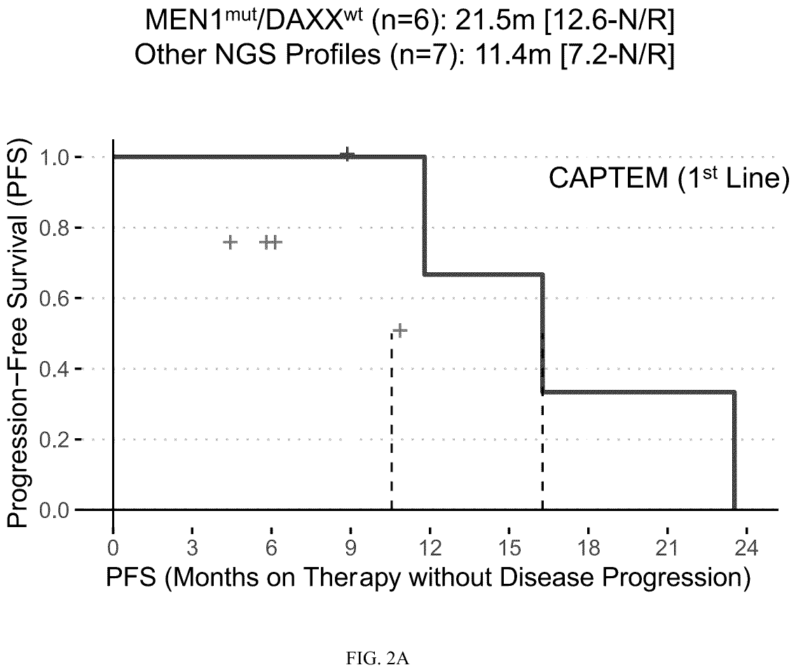

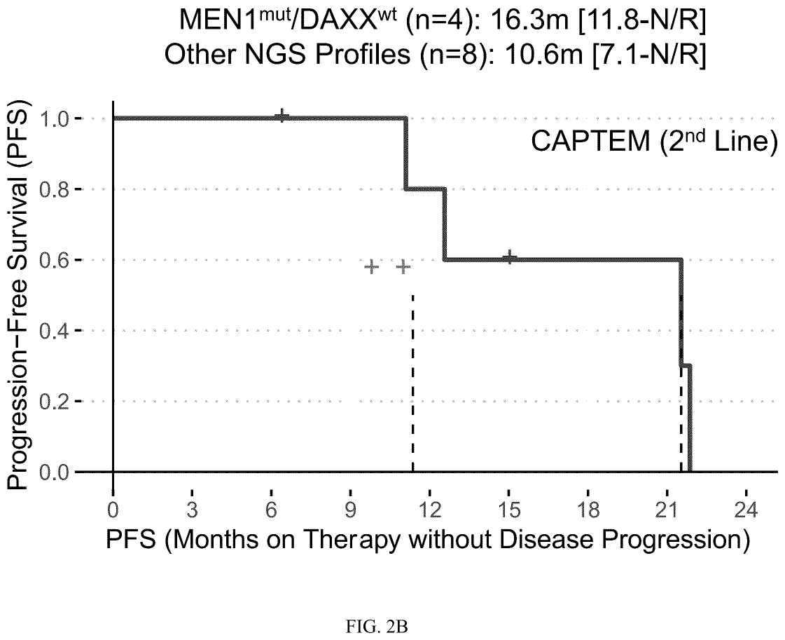

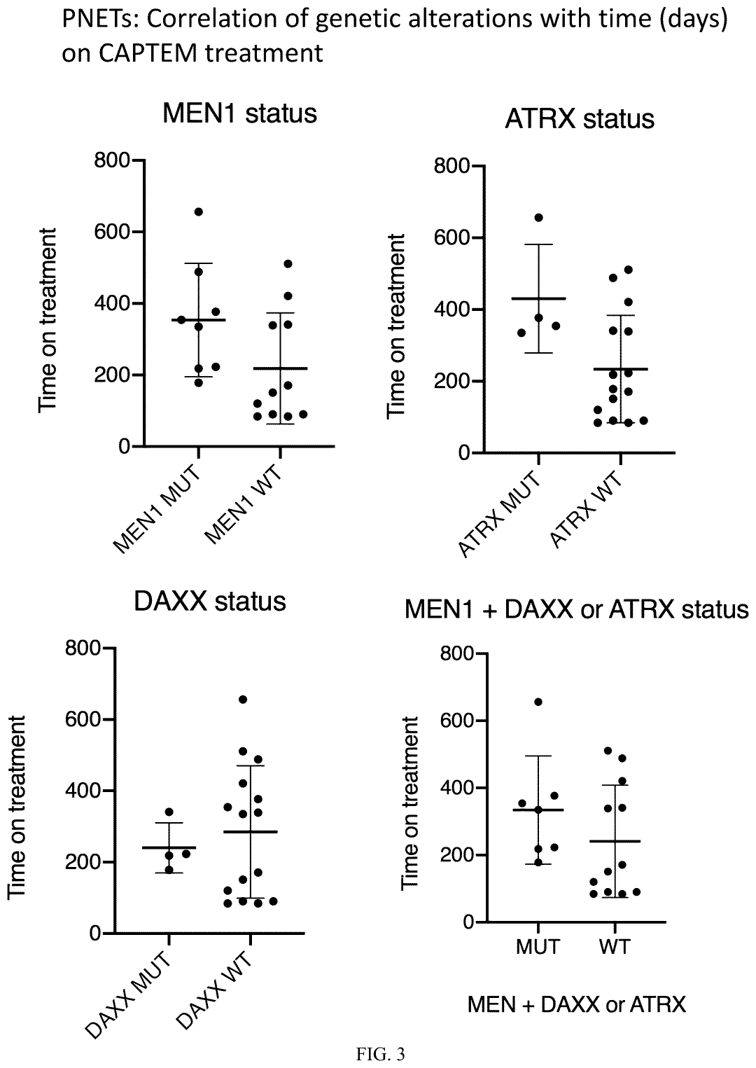

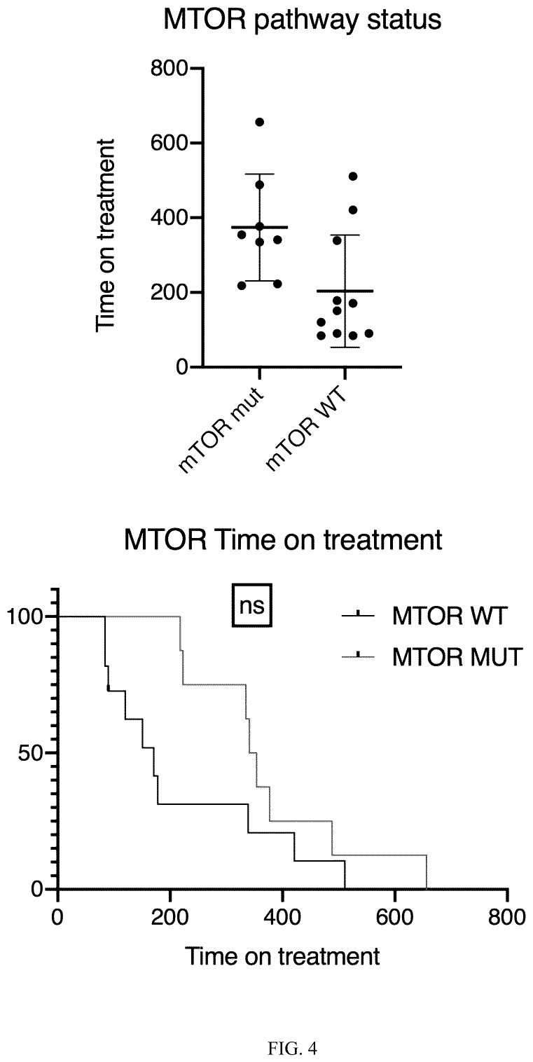

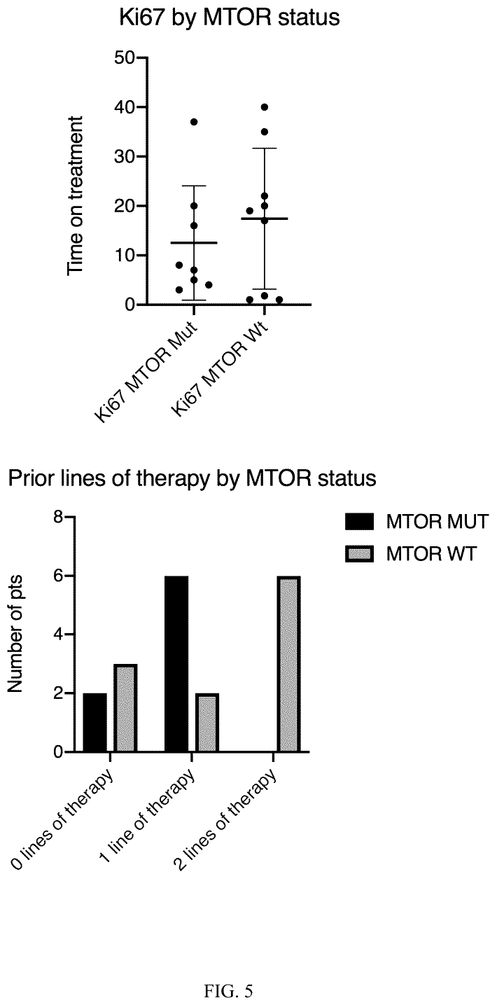

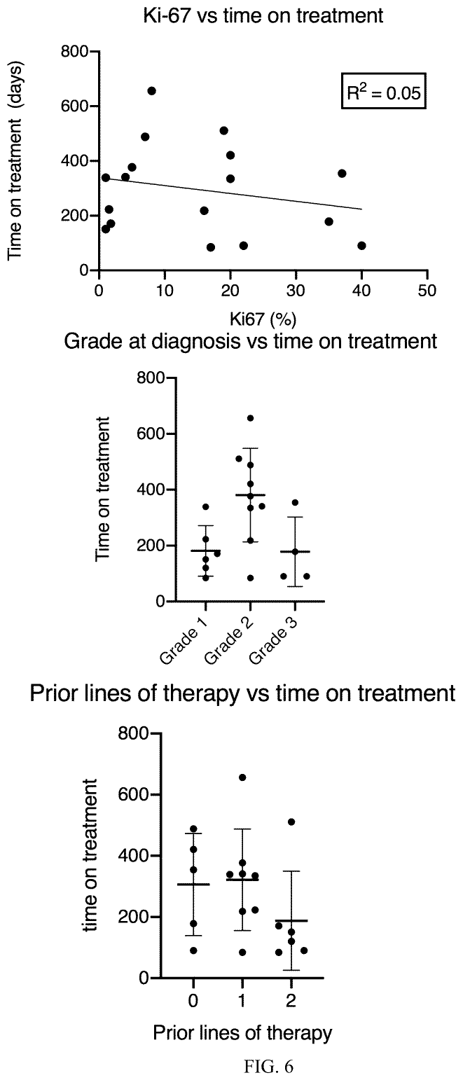

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

Pancreatic neuroendocrine neoplasms can be divided into tumors or carcinomas. In the Examples, we studied pancreatic neuroendocrine tumors (pNET or PNET). PNET is also known as islet cell tumors. There are two kinds of cells in the pancreas: endocrine pancreas cells and exocrine pancreas cells, wherein endocrine pancreas cells make several kinds of hormones and they cluster together in many small groups (islets) throughout the pancreas, hence endocrine pancreas cells being also called islet cells of islets of Langerhans. Tumors that form in islet cells are called islet cell tumors, pancreatic endocrine tumors, or pancreatic neuroendocrine tumors (pancreatic NETs). In contrast, exocrine pancreas cells make enzymes that are released into the small intestine to help the body digest food. Pancreatic NETs may be functional or nonfunctional: functional tumors make extra amounts of hormones, such as gastrin, insulin, and glucagon, that cause signs and symptoms, whereas nonfunctional tumors do not make extra amounts of hormones. Most pancreatic NETs are functional tumors.

PNET can be described by its grade (G) on how fast the tumor cells are growing and dividing, according to the World Health Organization. The grade can be measured in 2 ways: mitosis and Ki-67. A mitotic index is the number of dividing cells seen in 2 millimeters squared under a microscope. A Ki-67 index, measured via a histological stain, is an indicator of how quickly the tumor cells are multiplying, i.e., percentage of cells stained positive for Ki-67; and it has a range of 1 to 100 (out of 100). If there is a high percentage of cells in an area with Ki-67, it means that the cells are dividing rapidly. GX: Grade cannot be evaluated. G1: Mitotic index is less than 2, or Ki-67 index is less than 3 (i.e., Ki-67 index being 2 or less than 2). G2: Mitotic index is between 2 and 20, or Ki-67 index is 3 to 20. G3: Mitotic index is more than 20, or Ki-67 index is more than 20.

In some embodiments, the pancreatic neuroendocrine tumor to be treated is stage 0, stage I, stage II, stage III, or stage IV. In some embodiments, the pancreatic neuroendocrine tumor is metastatic pancreatic cancer.

Capecitabine is a type of chemotherapy called an anti-metabolite. The body changes capecitabine into a chemotherapy drug called fluorouracil. It stops cells from making and repairing DNA, thereby inhibiting the growth and proliferation of cancer cells. In some embodiments, capecitabine is administered in place of intravenous 5-FU primarily based on differences in toxicity and ease of administration. In other embodiments, a derivative of capecitabine is a metabolite of capecitabine, such as 5-FU, and hence the gene mutation status disclosed herein can also be used to monitor, prognose patients treated with 5-FU.

Temozolomide is also a type of chemotherapy. Temozolomide is an alkylating agent. Temozolomide hydrolyzes at physiological pH to 3-methyl-(triazen-1-yl)imidazole-4-carboxamide (MTIC). In some embodiments, a derivative of temozolomide is MTIC. In other embodiments, a derivative of temozolomide is dacarbazine, which is an analogue of temozolomide.

In various instances, the CAPTEM therapy is given orally to subjects in need thereof. In some instances, the CAPTEM therapy is given to subjects via injection into a vein or muscle. In some embodiments, capecitabine is given at about 750 mg/m² twice daily on days 1-14, and temozolomide is given at about 200 mg/m²/d on days 10-14 every 28 days. In some embodiments, capecitabine is given at about 675 mg/m² twice a day and temozolomide is given at about 180 mg/m². In some embodiments, capecitabine (or fluorouracil) is administered between 100-200, 200-300, 300-400, 400-500, 500-600, 600-700, 700-800, 800-900, or 900-1000 mg/m² of body surface area of a human, for one or two times a day for 1-5, 5-10, 10-15, or 15-20 days or a period of time, and then no capecitabine (or fluorouracil) is given for an interval of 1-10, 10-20, or 20-30 days before resuming administration of capecitabine (or fluorouracil). In some embodiments, temozolomide (or dacarbazine) is administered between 50-100, 100-150, 150-160, 160-170, 170-180, 180-190, 190-200, 200-210, 210-220, 220-230, 230-240, 240-250, 250-300, or 300-400 mg/m²/day for a period of time (e.g., 1, 2, 3, 4, 5, 6, 7, 8, 9, or 10 days), then paused for a period of time before it is re-administered. In some embodiments, capecitabine or fluorouracil and temozolomide or dacarbazine are administered sequentially, or only concurrently for some portion of a regimen and not the entire regimen. In other embodiments, capecitabine or fluorouracil and temozolomide or dacarbazine are administered on same days. In some embodiments, the CAPTEM therapy is given for 1, 2, 3, 4, 5, 6, 7, 8, 9, 10, 11, or 12 months, or longer. In some embodiments, the CAPTEM therapy is continued beyond 1 year per practitioner's discretion. In some embodiments, the CAPTEM therapy is given to post-surgery PNET patients. In some embodiments, the subject is also given prophylactic antiemetics before temozolomide, such as ondansetron 8 mg given 30 to 60 minutes before temozolomide.

A “derivative” is a compound produced from an original compound either directly or by modification or partial substitution of the original compound core or a compound that can at least in theory arise from another compound, if one atom is replaced with another atom or group of atoms. Different forms of compound derivatives include salts, isomers, analogues, metabolites or prodrugs, crystals or polymorphs, and solvates or hydrates.

“First-line therapy,” “first line therapy,” “first-line treatment,” or “first line treatment” are used interchangeably unless otherwise noted, and it is the first treatment given for a disease. If the first-line therapy doesn't cure the disease or it causes severe side effects, other treatment may be added or used instead. It may also be called induction therapy, primary therapy, and primary treatment.

“Second-line therapy,” “second line therapy,” “second-line treatment,” and “second line treatment” are used interchangeably unless otherwise noted. It is treatment that is given when initial treatment (first-line therapy) does not work or stops working.

Median progression-free survival refers to a time duration from the date of initiation of a particular therapy, herein CAPTEM in the Examples, until disease progression or patient death or change of therapy.

Overall survival refers to a time duration from the date of initial diagnosis of advanced, unresectable, or metastatic (henceforth summarized as advanced) disease at first presentation until the date of death or the last date the patient was known to be alive.

A mutation in a gene can include base substitution, deletion, and/or insertion, typically resulting in a functionally different protein compared to the wild-type gene or protein. Wild type genes, their nomenclature, and their sequences are available in publicly accessible database such as GENBANK®, an NIH genetic sequence database. In various implementations, a detected gene sequence other than the wild type sequence in this database is considered a mutation. In various implementations, a mutation is a pathogenic variant, which is in publicly assessable database such as GENBANK. In various embodiments, a mutation in a gene is detected via sequencing of mRNA of the gene, wherein an mRNA sequence other than the wild type sequence is considered a mutation. In various embodiments, wildtype MEN1 accession number is NM_130804; wildtype ATRX accession number is NM_000489; wildtype DAXX accession number is NM_001350; wildtype PIK3CA accession number is NM_006218; wildtype AKT1 accession number is NM_001014432; wildtype PTEN accession number is NM_000314; wildtype PIK3R1 accession number is NM_181523; wildtype PIK3R2 accession number is NM_005027; wildtype PIK3CG accession number is NM_001282426; wildtype PIK3CB accession number is NM_006219; wildtype PIK3CD accession number is NM_005026; wildtype MTOR accession number is NM_004958; and wildtype PIK3C2B accession number is NM_002646; and accession numbers are NCBI Reference Sequence numbers.

Homo sapiens gene MEN1 encodes menin, a tumor suppressor associated with a syndrome known as multiple endocrine neoplasia type 1.

Homo sapiens gene DAXXencodes death domain associated protein.

Homo sapiens gene ATRX (ATRX Chromatin Remodeler) encodes a protein containing an ATPase/helicase domain, which belongs to the SWI/SNF family of chromatin remodeling proteins.

“Statistically significant” generally means that the difference between two values has a p-value of ≤0.05, i.e., has a 95% or higher chance of representing a meaningful difference between the two values. In some embodiments, a statistical significant difference has a p-value of ≤0.01, i.e., has a 99% or higher chance of representing a meaningful difference between the two values.

The terms, “patient”, “individual” and “subject” are used interchangeably herein. A “subject” means a human or animal. Usually the animal is a vertebrate such as a primate, rodent, domestic animal or game animal. In an embodiment, the subject is mammal. The mammal can be a human, non-human primate, mouse, rat, dog, cat, horse, or cow, but are not limited to these examples. In various embodiments, the subject is human. In some embodiments, the subject has one or more metastatic sites of the PNET, such as in the liver or lung. Despite the frequent use of capecitabine and temozolomide (CAPTEM) to treat metastatic, well-differentiated pancreatic neuroendocrine tumors (PNETs), no reliable genomic predictors of response currently exist. We sought to determine whether the mutational status of MEN1, ATRX, DAXX, and the PI3K/AKT/mTOR pathway (e.g., AKT1, PTEN, PIK3R1, PIK3R2, PIK3CG, PIK3CB, PIK3CD, MTOR, and PIK3C2B) correlates with response to CAPTEM. Our findings confirm that patients with Men-1 mutations and those with ATRX wild-type preferentially benefit from this therapy of CAPTEM. This is potentially the first predictive biomarker discovered in pancreatic neuroendocrine tumors and could help stratify treatment approaches in this disease.

Various embodiments provide methods for treating a subject with pancreatic neuroendocrine tumor (PNET), and the methods include administering an effective amount of a composition comprising capecitabine or a derivative of capecitabine and/or temozolomide or a derivative of temozolomide to the subject detected with mutated MEN1 and wild-type DAXX in a biological sample of the subject. In some embodiments, the methods include administering an effective amount of a composition comprising capecitabine or a derivative of capecitabine and temozolomide or a derivative of temozolomide to the subject detected with mutated MEN1 and wild-type DAXX in a biological sample of the subject.

Various embodiments provide methods for selecting a subject with pancreatic neuroendocrine tumor (PNET) for CAPTEM therapy and treating the subject, and the methods include detecting mutated MEN1 and wild-type DAXX in a biological sample of the subject with the PNET, and administering the CAPTEM therapy to the subject, wherein the CAPTEM therapy comprises an effective amount of a composition comprising capecitabine or a derivative of capecitabine and/or temozolomide or a derivative of temozolomide. In some embodiments, the methods include detecting mutated MEN1 and wild-type DAXX in a biological sample of the subject with the PNET, and administering the CAPTEM therapy to the subject, wherein the CAPTEM therapy comprises an effective amount of a composition comprising capecitabine or a derivative of capecitabine and temozolomide or a derivative of temozolomide.

In various embodiments, administration of the CAPTEM therapy is based on an understanding that, after receiving the composition, the subject with the mutated MEN1 and the wild-type DAXX is likely to have a longer progression-free survival period than a control subject with the PNET but with wild-type MEN1 or mutated DAXX.

In some embodiments, the biological sample comprises a tumor cell or tissue of the PNET.

In some embodiments, the subject is further detected with mutated ATRX and/or mutated PTEN in the biological sample.

Some embodiments provide that detecting a gene expression pattern (including mutational profile) is performed by mRNA sequencing, preferably single-nuclei RNA sequence, and/or by DNA sequencing, for determination/detection of mutation and/or expression levels. In some embodiments, the detection comprises performing next-generation sequencing of DNA, RNA, or both of respective genes. In some embodiments, the detection comprises performing next-generation sequencing of mRNA of respective genes.

In some embodiments, the biological sample is obtained from the subject who has not received capecitabine or temozolomide before. Hence, the methods also provide a prognosis and/or identifies a subject responsive to the CAPTEM therapy, and may select the subject for CAPTEM therapy. In further embodiments, the methods identify subjects for receiving the CAPTEM therapy as a first-line therapy.

In some embodiments, the biological sample is obtained from the subject after the subject has received a prior administration of capecitabine and/or temozolomide. As such, the methods can provide information on whether the subject should continue CAPTEM therapy.

In some embodiments, the methods identify subjects for receiving the CAPTEM therapy as a second-line therapy. In some embodiments, a subject has received temozolomide (or dacarbazine) only, and the methods identify him/her for a combination therapy of capecitabine (or fluorouracil) and the temozolomide (or dacarbazine) if the subject's tumor sample is detected with mutated MEN1 gene and wildtype DAXX gene.

Other types of treatment than chemotherapy may be used for patients with PNET, including but are not limited to surgery to remove the tumor (e.g., enucleation, pancreatoduodenectomy, distal pancreatectomy, radiofrequency ablation, cryosurgical ablation, etc.), hormone therapy to remove hormones or block their action and stop cancer cells from growing (e.g., drugs, surgery, or radiation therapy to reduce production of hormones or block them from working), and hepatic arterial occlusion or chemoembolization (chemotherapy delivered during hepatic arterial occlusion).

Therefore, in some embodiments, the methods identify subjects for receiving the CAPTEM therapy as a second-line therapy, when first-line treatment such as surgery does not fully remove the tumor or the tumor recurs. In other embodiments, patients identified in the methods as unresponsive to the CAPTEM therapy or with poor prognostic outcome will receive another type of treatment, such as surgery, ¹⁷⁷Lu-Dotatate (a radiolabeled somatostatin analog), hormone therapy, hepatic arterial occlusion or chemoembolization.

In some embodiments, the CAPTEM therapy is provided in a composition that comprises a derivative of capecitabine and/or a derivative of temozolomide. In some embodiments, a derivative of capecitabine is a metabolite of capecitabine, such as 5-fluorouracil. In some embodiments, a derivative of capecitabine is a salt of capecitabine. In some embodiments, a derivative of temozolomide is a salt of temozolomide. In some embodiments, a derivative of temozolomide is its metabolite such as MTIC, or its analogue such as dacarbazine. In some embodiments, a CAPTEM therapy includes a combination of (1) capecitabine or fluorouracil, and (2) temozolomide or dacarbazine.

In various embodiments, the subject is a human. In various embodiments, the subject has PNET. In some embodiments, the subject has other neuroendocrine tumor (NET) subtypes, including lung and/or thymus. In some embodiments, the subject has had no prior treatment with temozolomide, dacarbazine, capecitabine, or fluorouracil.

Various embodiments provide methods which include performing gene sequencing for DNA, RNA, or both of one or more genes comprising MEN1, DAXX, ATRX, and PTEN, and further optionally one or more of AKT1, PTEN, PIK3R1, PIK3R2, PIK3CG, PIK3CB, PIK3CD, MTOR, and PIK3C2B, in the biological sample, and detecting presence or absence of a mutation in each of the one or more genes, wherein the biological sample comprises a tumor cell or tissue of the PNET.

In some embodiments, the one or more genes in the gene sequencing include MEN1 and DAXX. In some embodiments, the methods of assaying the biological sample from a subject with PNET further includes selecting the subject for a CAPTEM therapy, wherein the subject is detected with a presence of mutated MEN1 and an absence of mutated DAXX.

Further embodiments provide methods of providing prognosis to a subject receiving a CAPTEM therapy, and/or determining if the subject is predicted to respond to the CAPTEM therapy, wherein the subject has pancreatic neuroendocrine tumor (PNET) and the CAPTEM therapy comprises capecitabine or a derivative of capecitabine and/or temozolomide or a derivative of temozolomide; and the methods include: performing gene sequencing for DNA, RNA, or both of genes comprising MEN1 and DAXX in a biological sample of the subject, to detect presence or absence of a mutation in each of the genes, said biological sample comprises a tumor cell or tissue of the PNET.

In some embodiments, the subject is prognosed with a longer progression-free survival period after receiving the CAPTEM therapy, and/or the subject is indicated to be better responsive to the CAPTEM therapy, when the subject is detected with the presence of mutated MEN1 and wild-type DAXX, compared to a first control subject with the PNET but having wild-type MEN1 and/or mutated DAXX in a PNET cell or tissue of the first control subject.

In some embodiments, the subject is prognosed with a shorter progression-free survival period after receiving the CAPTEM therapy, and/or the subject is indicated to be less responsive to the CAPTEM therapy, when the subject is detected with wild-type MEN1 and/or the presence of mutated DAXX, compared to a second control subject with the PNET but having mutated MEN1 and wild-type DAXX in a PNET cell or tissue of the second control subject.

Additional embodiments provide a kit, suitable for use in assaying a biological sample from a subject with or suspected of having PNET, wherein the kit includes one or more primer nucleotide sequences for specifically sequencing each of genes MEN1 and DAXX, and also optionally ATRX and PTEN. Further embodiments provide a kit, suitable for use in assaying a biological sample from a subject in need thereof (e.g., having PNET or desiring a prognosis of PNET outcome), wherein the kit includes polynucleotides, peptides, or small molecule reagents that bind to the full range or at least 50%, 60%, 70%, 80%, or 90% of the range of each coordinates for each gene in Table 1 or for at least 1, 2, 3, 4, 5 or more coordinates of each different genes in Table 1. In some embodiments, a kit includes nucleic acid sequences that bind to each region shown by starting and ending coordinates in Table 1.

TABLE 1
Starting and ending coordinates of genes assayed in Examples.

| | Starting | Ending | Gene ID |
|---|---|---|---|
| chr6 | 33286485 | 33286609 | GENE_ID = DAXX |
| chr6 | 33286758 | 33286888 | GENE_ID = DAXX |
| chr6 | 33286884 | 33287001 | GENE_ID = DAXX |
| chr6 | 33287133 | 33287268 | GENE_ID = DAXX |
| chr6 | 33287249 | 33287378 | GENE_ID = DAXX |
| chr6 | 33287357 | 33287485 | GENE_ID = DAXX |
| chr6 | 33287469 | 33287585 | GENE_ID = DAXX |
| chr6 | 33287562 | 33287678 | GENE_ID = DAXX |
| chr6 | 33287751 | 33287853 | GENE_ID = DAXX |
| chr6 | 33287829 | 33287946 | GENE_ID = DAXX |
| chr6 | 33287927 | 33288040 | GENE_ID = DAXX |
| chr6 | 33288136 | 33288245 | GENE_ID = DAXX |
| chr6 | 33288233 | 33288343 | GENE_ID = DAXX |
| chr6 | 33288327 | 33288455 | GENE_ID = DAXX |
| chr6 | 33288479 | 33288591 | GENE_ID = DAXX |
| chr6 | 33288566 | 33288694 | GENE_ID = DAXX |
| chr6 | 33288686 | 33288817 | GENE_ID = DAXX |
| chr6 | 33288801 | 33288916 | GENE_ID = DAXX |
| chr6 | 33288897 | 33289027 | GENE_ID = DAXX |
| chr6 | 33289015 | 33289150 | GENE_ID = DAXX |
| chr6 | 33289138 | 33289268 | GENE_ID = DAXX |
| chr6 | 33289235 | 33289340 | GENE_ID = DAXX |
| chr6 | 33289326 | 33289458 | GENE_ID = DAXX |
| chr6 | 33289454 | 33289580 | GENE_ID = DAXX |
| chr6 | 33289530 | 33289649 | GENE_ID = DAXX |
| chr6 | 33289615 | 33289747 | GENE_ID = DAXX |
| chr6 | 33290598 | 33290729 | GENE_ID = DAXX |
| chr10 | 89623658 | 89623799 | GENE_ID = PTEN |
| chr10 | 89623807 | 89623945 | GENE_ID = PTEN |
| chr10 | 89623876 | 89624006 | GENE_ID = PTEN |
| chr10 | 89623992 | 89624082 | GENE_ID = PTEN |
| chr10 | 89624059 | 89624193 | GENE_ID = PTEN |
| chr10 | 89624163 | 89624310 | GENE_ID = PTEN |
| chr10 | 89653766 | 89653840 | GENE_ID = PTEN |
| chr10 | 89653837 | 89653959 | GENE_ID = PTEN |
| chr10 | 89685258 | 89685355 | GENE_ID = PTEN |
| chr10 | 89690782 | 89690877 | GENE_ID = PTEN |
| chr10 | 89692717 | 89692835 | GENE_ID = PTEN |
| chr10 | 89692819 | 89692950 | GENE_ID = PTEN |
| chr10 | 89692922 | 89693038 | GENE_ID = PTEN |
| chr10 | 89711802 | 89711933 | GENE_ID = PTEN |
| chr10 | 89711903 | 89712000 | GENE_ID = PTEN |
| chr10 | 89711979 | 89712106 | GENE_ID = PTEN |
| chr10 | 89717528 | 89717649 | GENE_ID = PTEN |
| chr10 | 89717629 | 89717746 | GENE_ID = PTEN |
| chr10 | 89717740 | 89717858 | GENE_ID = PTEN |
| chr10 | 89720576 | 89720694 | GENE_ID = PTEN |
| chr10 | 89720686 | 89720823 | GENE_ID = PTEN |
| chr10 | 89720769 | 89720908 | GENE_ID = PTEN |
| chr10 | 89725031 | 89725171 | GENE_ID = PTEN |
| chr10 | 89725171 | 89725309 | GENE_ID = PTEN |
| chr11 | 64571784 | 64571922 | GENE_ID = MEN1 |
| chr11 | 64571920 | 64572060 | GENE_ID = MEN1 |
| chr11 | 64572007 | 64572126 | GENE_ID = MEN1 |
| chr11 | 64572116 | 64572254 | GENE_ID = MEN1 |
| chr11 | 64572222 | 64572360 | GENE_ID = MEN1 |
| chr11 | 64572485 | 64572624 | GENE_ID = MEN1 |
| chr11 | 64572611 | 64572691 | GENE_ID = MEN1 |
| chr11 | 64573041 | 64573173 | GENE_ID = MEN1 |
| chr11 | 64573173 | 64573283 | GENE_ID = MEN1 |
| chr11 | 64573660 | 64573789 | GENE_ID = MEN1 |
| chr11 | 64573723 | 64573862 | GENE_ID = MEN1 |
| chr11 | 64574458 | 64574574 | GENE_ID = MEN1 |
| chr11 | 64574559 | 64574688 | GENE_ID = MEN1 |
| chr11 | 64574676 | 64574795 | GENE_ID = MEN1 |
| chr11 | 64574993 | 64575121 | GENE_ID = MEN1 |
| chr11 | 64575097 | 64575220 | GENE_ID = MEN1 |
| chr11 | 64575331 | 64575465 | GENE_ID = MEN1 |
| chr11 | 64575461 | 64575591 | GENE_ID = MEN1 |
| chr11 | 64577080 | 64577203 | GENE_ID = MEN1 |
| chr11 | 64577184 | 64577321 | GENE_ID = MEN1 |
| chr11 | 64577245 | 64577382 | GENE_ID = MEN1 |
| chr11 | 64577360 | 64577451 | GENE_ID = MEN1 |
| chr11 | 64577410 | 64577548 | GENE_ID = MEN1 |
| chr11 | 64577497 | 64577636 | GENE_ID = MEN1 |
| chrX | 76763768 | 76763890 | GENE_ID = ATRX |
| chrX | 76763831 | 76763949 | GENE_ID = ATRX |
| chrX | 76763940 | 76764067 | GENE_ID = ATRX |
| chrX | 76764026 | 76764140 | GENE_ID = ATRX |
| chrX | 76776258 | 76776374 | GENE_ID = ATRX |
| chrX | 76776362 | 76776457 | GENE_ID = ATRX |
| chrX | 76776857 | 76776947 | GENE_ID = ATRX |
| chrX | 76776919 | 76777045 | GENE_ID = ATRX |
| chrX | 76777679 | 76777804 | GENE_ID = ATRX |
| chrX | 76777796 | 76777909 | GENE_ID = ATRX |
| chrX | 76778689 | 76778795 | GENE_ID = ATRX |
| chrX | 76778752 | 76778873 | GENE_ID = ATRX |
| chrX | 76778842 | 76778955 | GENE_ID = ATRX |
| chrX | 76812893 | 76813019 | GENE_ID = ATRX |
| chrX | 76812962 | 76813081 | GENE_ID = ATRX |
| chrX | 76813066 | 76813173 | GENE_ID = ATRX |
| chrX | 76814124 | 76814235 | GENE_ID = ATRX |
| chrX | 76814207 | 76814311 | GENE_ID = ATRX |
| chrX | 76814300 | 76814408 | GENE_ID = ATRX |
| chrX | 76829622 | 76829747 | GENE_ID = ATRX |
| chrX | 76829745 | 76829844 | GENE_ID = ATRX |
| chrX | 76845222 | 76845334 | GENE_ID = ATRX |
| chrX | 76845310 | 76845392 | GENE_ID = ATRX |
| chrX | 76845385 | 76845492 | GENE_ID = ATRX |
| chrX | 76849110 | 76849217 | GENE_ID = ATRX |
| chrX | 76849194 | 76849317 | GENE_ID = ATRX |
| chrX | 76849272 | 76849392 | GENE_ID = ATRX |
| chrX | 76854804 | 76854920 | GENE_ID = ATRX |
| chrX | 76854884 | 76854978 | GENE_ID = ATRX |
| chrX | 76854959 | 76855079 | GENE_ID = ATRX |
| chrX | 76855183 | 76855268 | GENE_ID = ATRX |
| chrX | 76855251 | 76855368 | GENE_ID = ATRX |
| chrX | 76855876 | 76855940 | GENE_ID = ATRX |
| chrX | 76855904 | 76856007 | GENE_ID = ATRX |
| chrX | 76855991 | 76856067 | GENE_ID = ATRX |
| chrX | 76872003 | 76872114 | GENE_ID = ATRX |
| chrX | 76872091 | 76872179 | GENE_ID = ATRX |
| chrX | 76872170 | 76872252 | GENE_ID = ATRX |
| chrX | 76874226 | 76874321 | GENE_ID = ATRX |
| chrX | 76874280 | 76874367 | GENE_ID = ATRX |
| chrX | 76874362 | 76874483 | GENE_ID = ATRX |
| chrX | 76875823 | 76875925 | GENE_ID = ATRX |
| chrX | 76875921 | 76875993 | GENE_ID = ATRX |
| chrX | 76875988 | 76876074 | GENE_ID = ATRX |
| chrX | 76888633 | 76888742 | GENE_ID = ATRX |
| chrX | 76888715 | 76888841 | GENE_ID = ATRX |
| chrX | 76888807 | 76888932 | GENE_ID = ATRX |
| chrX | 76888955 | 76889073 | GENE_ID = ATRX |
| chrX | 76889065 | 76889174 | GENE_ID = ATRX |
| chrX | 76889159 | 76889284 | GENE_ID = ATRX |
| chrX | 76890008 | 76890110 | GENE_ID = ATRX |
| chrX | 76890087 | 76890167 | GENE_ID = ATRX |
| chrX | 76890146 | 76890268 | GENE_ID = ATRX |
| chrX | 76891365 | 76891489 | GENE_ID = ATRX |
| chrX | 76891478 | 76891574 | GENE_ID = ATRX |
| chrX | 76907568 | 76907672 | GENE_ID = ATRX |
| chrX | 76907629 | 76907755 | GENE_ID = ATRX |
| chrX | 76907736 | 76907865 | GENE_ID = ATRX |
| chrX | 76909510 | 76909614 | GENE_ID = ATRX |
| chrX | 76909612 | 76909684 | GENE_ID = ATRX |
| chrX | 76909663 | 76909763 | GENE_ID = ATRX |
| chrX | 76912002 | 76912121 | GENE_ID = ATRX |
| chrX | 76912109 | 76912230 | GENE_ID = ATRX |
| chrX | 76918831 | 76918950 | GENE_ID = ATRX |
| chrX | 76918947 | 76919071 | GENE_ID = ATRX |
| chrX | 76920068 | 76920179 | GENE_ID = ATRX |
| chrX | 76920155 | 76920251 | GENE_ID = ATRX |
| chrX | 76920240 | 76920343 | GENE_ID = ATRX |
| chrX | 76931690 | 76931779 | GENE_ID = ATRX |
| chrX | 76931763 | 76931871 | GENE_ID = ATRX |
| chrX | 76936921 | 76937038 | GENE_ID = ATRX |
| chrX | 76937029 | 76937129 | GENE_ID = ATRX |
| chrX | 76937114 | 76937193 | GENE_ID = ATRX |
| chrX | 76937181 | 76937264 | GENE_ID = ATRX |
| chrX | 76937240 | 76937339 | GENE_ID = ATRX |
| chrX | 76937310 | 76937428 | GENE_ID = ATRX |
| chrX | 76937402 | 76937522 | GENE_ID = ATRX |
| chrX | 76937496 | 76937608 | GENE_ID = ATRX |
| chrX | 76937571 | 76937688 | GENE_ID = ATRX |
| chrX | 76937686 | 76937808 | GENE_ID = ATRX |
| chrX | 76937797 | 76937917 | GENE_ID = ATRX |
| chrX | 76937855 | 76937983 | GENE_ID = ATRX |
| chrX | 76938013 | 76938123 | GENE_ID = ATRX |
| chrX | 76938027 | 76938151 | GENE_ID = ATRX |
| chrX | 76938175 | 76938301 | GENE_ID = ATRX |
| chrX | 76938291 | 76938405 | GENE_ID = ATRX |
| chrX | 76938387 | 76938511 | GENE_ID = ATRX |
| chrX | 76938520 | 76938609 | GENE_ID = ATRX |
| chrX | 76938604 | 76938728 | GENE_ID = ATRX |
| chrX | 76938713 | 76938828 | GENE_ID = ATRX |
| chrX | 76938831 | 76938949 | GENE_ID = ATRX |
| chrX | 76938945 | 76939063 | GENE_ID = ATRX |
| chrX | 76939040 | 76939158 | GENE_ID = ATRX |
| chrX | 76939147 | 76939271 | GENE_ID = ATRX |
| chrX | 76939251 | 76939373 | GENE_ID = ATRX |
| chrX | 76939343 | 76939482 | GENE_ID = ATRX |
| chrX | 76939446 | 76939566 | GENE_ID = ATRX |
| chrX | 76939537 | 76939682 | GENE_ID = ATRX |
| chrX | 76939665 | 76939787 | GENE_ID = ATRX |
| chrX | 76939775 | 76939893 | GENE_ID = ATRX |
| chrX | 76939882 | 76940006 | GENE_ID = ATRX |
| chrX | 76939999 | 76940109 | GENE_ID = ATRX |
| chrX | 76940403 | 76940511 | GENE_ID = ATRX |
| chrX | 76944240 | 76944365 | GENE_ID = ATRX |
| chrX | 76944350 | 76944419 | GENE_ID = ATRX |
| chrX | 76944398 | 76944496 | GENE_ID = ATRX |
| chrX | 76949281 | 76949402 | GENE_ID = ATRX |
| chrX | 76949389 | 76949505 | GENE_ID = ATRX |
| chrX | 76951977 | 76952090 | GENE_ID = ATRX |
| chrX | 76952066 | 76952164 | GENE_ID = ATRX |
| chrX | 76952139 | 76952262 | GENE_ID = ATRX |
| chrX | 76952989 | 76953086 | GENE_ID = ATRX |
| chrX | 76953068 | 76953189 | GENE_ID = ATRX |
| chrX | 76954041 | 76954152 | GENE_ID = ATRX |
| chrX | 76972541 | 76972629 | GENE_ID = ATRX |
| chrX | 76972614 | 76972696 | GENE_ID = ATRX |
| chrX | 76972688 | 76972781 | GENE_ID = ATRX |
| chrX | 77041424 | 77041552 | GENE_ID = ATRX |

Determining the presence of a particular variance (e.g., mutation) or plurality of variances in a particular gene in a patient can be performed in a variety of ways. In various embodiments, the detection of the presence or absence of at least one variance involves amplifying a segment of nucleic acid including at least one of the at least one variances. Preferably a segment of nucleic acid to be amplified is 500 nucleotides or less in length, more preferably 100 nucleotides or less, and most preferably 45 nucleotides or less. Also, preferably the amplified segment or segments includes a plurality of variances, or a plurality of segments of a gene or of a plurality of genes. In other embodiments, e.g., where a haplotype is to be determined, the segment of nucleic acid is at least 500 nucleotides in length, or at least 2 kb in length, or at least 5 kb in length.

In preferred embodiments, determining the presence of a set of variances in a specific gene includes a haplotyping test that involves allele specific amplification of a large DNA segment of no greater than 25,000 nucleotides, preferably no greater than 10,000 nucleotides and most preferably no greater than 5,000 nucleotides. Alternatively one allele may be enriched by methods other than amplification prior to determining genotypes at specific variant positions on the enriched allele as a way of determining haplotypes. Preferably the determination of the presence or absence of a haplotype involves determining the sequence of the variant sites by methods such as chain terminating DNA sequencing or minisequencing, or by oligonucleotide hybridization or by mass spectrometry.

In preferred embodiments, determining the presence or absence of the at least one variance involves sequencing at least one nucleic acid sample. The sequencing involves sequencing of a portion or portions of a gene and/or portions of a plurality of genes which includes at least one variance site, and may include a plurality of such sites. Preferably, the portion is 500 nucleotides or less in length, more preferably 200 or 100 nucleotides or less, and most preferably 45 nucleotides or less in length. Such sequencing can be carried out by various methods recognized by those skilled in the art, including use of dideoxy termination methods (e.g., using dye-labeled dideoxy nucleotides) and the use of mass spectrometric methods. In addition, mass spectrometric methods may be used to determine the nucleotide present at a variance site. In preferred embodiments in which a plurality of variances is determined, the plurality of variances can constitute a haplotype or collection of haplotypes. Preferably the methods for determining genotypes or haplotypes are designed to be sensitive to all the common genotypes or haplotypes present in the population being studied (for example, a clinical trial population).

In other preferred embodiments, the detection of the presence or absence of the at least one variance involves contacting a nucleic acid sequence corresponding to one of the genes identified above or a product of such a gene with a probe. The probe is able to distinguish a particular form of the gene or gene product or the presence or a particular variance or variances, e.g., by differential binding or hybridization. Thus, exemplary probes include nucleic acid hybridization probes, peptide nucleic acid probes, nucleotide-containing probes which also contain at least one nucleotide analog, and antibodies, e.g., monoclonal antibodies, and other probes as discussed herein. Variables can be adjusted to optimize the discrimination between two variant forms of a gene, including changes in salt concentration, temperature, pH and addition of various compounds that affect the differential affinity of GC vs. AT base pairs, such as tetramethyl ammonium chloride. (See Current Protocols in Molecular Biology by F. M. Ausubel, R. Brent, R. E. Kingston, D. D. Moore, J. D. Seidman, K. Struhl, and V. B. Chanda (editors, John Wiley & Sons.)

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Genomic Correlates of Response to Capecitabine and Temozolomide (CAPTEM) in Pancreatic Neuroendocrine Tumors

Capecitabine plus temozolomide (CAPTEM) is commonly used to treat advanced/metastatic pancreatic neuroendocrine tumors (PNETs). However, no reliable genomic predictors of increased benefit from CAPTEM currently exist. PNETs commonly harbor mutations in MEN1, ATRX, DAXX, and PI3K/AKT/mTOR pathway genes. We hypothesized that specific mutational profiles in genes such as one or more of MEN1, ATRX, DAXX, AKT1, PTEN, PIK3R1, PIK3R2, PIK3CG, PIK3CB, PIK3CD, MTOR, and PIK3C2B in PNETs may correlate with response to CAPTEM.

A retrospective cohort (N=95) of PNET cases seen at Cedars-Sinai Medical Center or from Perthera's Real-World Evidence (RWE) Database (Pishvaian et al. The Lancet Oncology. March 2020; PMID: 32135080) included 23 patients who were treated with CAPTEM in 1st or 2nd line and who had tumor next-generation sequencing (NGS) results available. CAPTEM treatment was administered as capecitabine at 750 mg/m² by mouth twice a day for days 1-14 and temozolomide 200 mg/m² by mouth once daily on days 10-14, repeated every 28 days or monthly. Genomic alterations were correlated with progression-free survival (PFS) on CAPTEM using multivariate Cox regression analysis. A simplified genomic subgrouping based on MEN1/DAXX mutational status was defined for downstream analyses. Differences in PFS outcomes by MEN1^{mut}/DAXX^{wt} status and potential confounders (e.g., line of therapy) were analyzed using univariate and multivariate Cox regression.

We analyzed 23 PNET patients, 4 (17.4) of whom had documented functional tumors. We identified MEN1 mutations as positively associated with CAPTEM response, but this effect was less pronounced for the subset with co-occurring DAXX mutations. With and without accounting for line of therapy, we found that PFS on CAPTEM was significantly longer in MEN1-mutated, DAXX-wildtype tumors compared to other mutation profiles (P<0.01, see Table 2), wherein other mutation profiles are MEN1-wild type or DAXX-mutated. ATRX (67%) and PTEN (330%) alterations were also enriched in the MEN1-mutated/DAXX-wildtype subset; however, other P3K/AKT/mTOR alterations were common across all MEN1-mutated cases.

TABLE 2
Exploratory PFS analyses on 1st/2nd line CAPTEM comparing
MEN1-mutated/DAXX-wildtype PNETs versus other genomic profiles.

| | | | | Line of |
|---|---|---|---|---|
| | | Univariate | Multivariate | Therapy |
| | | p-value | p-value | p-value |
| | mPFS | (HR | (HR | (HR |
| PNET Subset | [95% CI] | [95% CI]) | [95% CI]) | [95% CI]) |
| MEN1-mutated | 16.3 m | 0.009932 | 0.009722 | 0.6245 |
| & DAXX-wildtype | [12.6-N/R] | (0.16 | (0.14 | (0.75 |
| (n = 9) | | [0.04-0.64]) | [0.03-0.63]) | [0.24-2.33]) |
| MEN1-wildtype | 7.4 m | | | |
| or DAXX-mutated | [7.1-N/R] | | | |
| (n = 14) | | | | |

TABLE 3
Cox regression analyses evaluating whether MEN1^{mut}/DAXX^{wt}
PNETs exhibit significantly longer PFS on CAPTEM
with and without taking into account line of therapy.

| | | Hazard | | Hazard |
|---|---|---|---|---|
| | Univariate | Ratio | Multivariate | Ratio |
| | Cox | (HR) | Cox | (HR) |
| | Significance | [95% Conf. | Significance | [95% Conf. |
| PFS Strata | (p) | Interval] | (p) | Interval] |
| MEN1^{mut}/ | p = 0.0094 | HR = 0.16 | p = 0.0097 | HR = 0.16 |
| DAXX^{wt} | | [0.04-0.64] | | [0.04-0.64] |
| vs Other NGS | | | | |
| Profiles | | | | |
| 1^{st} Line | p = 0.68 | HR = 0.8 | p = 0.86 | HR = 0.91 |
| CAPTEM vs | | [0.27-2.34] | | [0.32-2.58] |
| 2^{nd} Line | | | | |
| CAPTEM | | | | |

Therefore, we provide a new genomic signature (MEN1 mut/DAXX wt) that correlates with relative PNET response to CAPTEM therapy. MEN1-mut/DAXX-wt status correlates with longer PFS on CAPTEM in pancreatic neuroendocrine tumors. Prospective validation of these may further take into account other therapies, histopathologic factors, and other genomic correlates.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A method of treating a subject with pancreatic neuroendocrine tumor (PNET), comprising:
administering an effective amount of a composition comprising capecitabine or a derivative of capecitabine and temozolomide or a derivative of temozolomide to the subject detected with mutated MEN1 and wild-type DAXX in a biological sample of the subject.

2. The method of claim 1, for selecting the subject with the PNET for CAPTEM therapy and treating the subject, wherein the method further comprises:
detecting mutated MEN1 and wild-type DAXX in the biological sample of the subject with the PNET, and
administering the CAPTEM therapy to the subject,
wherein the CAPTEM therapy comprises an effective amount of a composition comprising capecitabine or a derivative of capecitabine and temozolomide or a derivative of temozolomide.

3. The method of claim 1, wherein the administration is based on an understanding that, after receiving the composition, the subject with the mutated MEN1 and the wild-type DAXX is likely to have a longer progression-free survival period than a control subject with the PNET but with wild-type MEN1 or mutated DAXX.

4. The method of claim 1, wherein the biological sample comprises a tumor cell or tissue of the PNET.

5. The method of claim 1, wherein the subject is further detected with mutated ATRX and/or mutated PTEN in the biological sample.

6. The method of claim 1, wherein the detection comprises performing next-generation sequencing of DNA, RNA, or both of respective genes.

7. The method of claim 1, wherein the biological sample is obtained from the subject who has not received capecitabine or temozolomide before.

8. The method of claim 1, wherein the biological sample is obtained from the subject after the subject has received a prior administration of capecitabine and/or temozolomide.

9. The method of claim 1, wherein the composition comprises the derivative of capecitabine and/or the derivative of temozolomide.

10. The method of claim 9, wherein the derivative of capecitabine is a metabolite of capecitabine, and the metabolite of capecitabine comprises 5-fluorouracil.

11. The method of claim 9, wherein the derivative of capecitabine is a salt of capecitabine.

12. The method of claim 9, wherein the derivative of temozolomide is a salt of temozolomide.

13. The method of claim 1, wherein the subject is a human.

14. A method of assaying a biological sample from a subject with pancreatic neuroendocrine tumor (PNET), comprising:
performing gene sequencing for DNA, RNA, or both of one or more genes comprising MEN1, DAXX, ATRX, and PTEN in the biological sample, and
detecting presence or absence of a mutation in each of the one or more genes,
wherein the biological sample comprises a tumor cell or tissue of the PNET.

15. The method of claim 14, wherein the one or more genes comprises the MEN1 and DAXX.

16. The method of claim 15, further comprising selecting the subject for a CAPTEM therapy, wherein the subject is detected with a presence of mutated MEN1 and an absence of mutated DAXX, and wherein the CAPTEM therapy comprises capecitabine or a derivative of capecitabine and temozolomide or a derivative of temozolomide.

17. The method of claim 14, wherein the subject has been detected with a mutation in the MEN1 gene, and the performing of gene sequencing comprises performing the gene sequencing of one or more of DAXX, ATRX, and PTEN of the subject.

18. The method of claim 15, further comprising providing prognosis to the subject, said subject receiving a CAPTEM therapy, and/or determining if the subject is predicted to respond to the CAPTEM therapy, wherein the CAPTEM therapy comprises capecitabine or a derivative of capecitabine and temozolomide or a derivative of temozolomide, wherein the subject is prognosed with a longer progression-free survival period after receiving the CAPTEM therapy, and/or the subject is indicated to be better responsive to the CAPTEM therapy, when the subject is detected with the presence of mutated MEN1 and wild-type DAXX, compared to a first control subject with the PNET but having mutated MEN1 and mutated DAXX or having wild-type MEN1 or having mutated DAXX in a PNET cell or tissue of the first control subject;
or wherein the subject is prognosed with a shorter progression-free survival period after receiving the CAPTEM therapy, and/or the subject is indicated to be less responsive to the CAPTEM therapy, when the subject is detected with mutated MEN1 and mutated DAXX or detected with wild-type MEN1 or detected with mutated DAXX, compared to a second control subject with the PNET but having mutated MEN1 and wild-type DAXX in a PNET cell or tissue of the second control subject.

19. A method of treating a subject having pancreatic neuroendocrine tumor, wherein the subject is detected with wild-type MEN1 gene and/or mutated DAXX gene in a tumor sample, the method comprising: performing surgery to remove the tumor, providing hormone therapy, and/or performing hepatic arterial occlusion or chemoembolization, to the subject detected with the wild-type MEN1 gene and/or the mutated DAXX gene.

20. The method of claim 19, wherein the method discontinues or does not include administering a CAPTEM therapy to the subject, wherein the CAPTEM therapy comprises a combination of capecitabine or a derivative of capecitabine and temozolomide or a derivative of temozolomide.
